(19) United States
(12) Patent Application Publication
(10) Pub. No.: US 2020/0154799 A1
(43) Pub. Date: May 21, 2020

(54) NECK GAITER SYSTEM

(71) Applicant: Tate Technology, LLC, Pacific Palisades, CA (US)

(72) Inventor: Jenny T. Morgan, Pacific Palisades, CA (US)

(21) Appl. No.: 16/688,852

(22) Filed: Nov. 19, 2019

Related U.S. Application Data
(60) Provisional application No. 62/769,955, filed on Nov. 20, 2018.

(51) Int. Cl. A41D 13/05; A61B 5/11; A61B 5/00

(52) U.S. Cl. CPC A41D 13/0512; A61B 5/1112; A61B 2562/0219; A61B 5/6822; A61B 5/002

(57) ABSTRACT

A neck gaiter system configured to be received on the neck of a wearer that includes a gaiter member that includes a neck portion and a clavicle portion, a sensor system that includes at least one gyro sensor positioned on the gaiter member, an actuation system in electrical communication with the sensor system, and a bladder positioned on at least the neck portion of the gaiter member. The bladder is in fluid communication with the actuation system. When the sensor system senses a predetermined rotational change, the actuation system inflates the bladder.

### CROSS REFERENCE TO RELATED APPLICATIONS

This application claims the benefit of U.S. Provisional Application No. 62/769,955, filed Nov. 20, 2018, the entirety of which is incorporated herein by reference.

### FIELD OF THE INVENTION

This invention relates generally to a disposable aspirated/inflatable safety detached neck collar/gaiter protection system designed to protect the human neck from injury resulting from sports impacts with a digital gyroscope and impact crash/force using accelerometers and machine learning with pre-set algorithms, plus bio-metric sensor arrangement system in which acceleration, angular, rotational, extension or flexion motion resulting from over rotation or unintended falling off of equipment or animals requires deployment of an apparatus protection device.

### BACKGROUND OF THE INVENTION

Traditional or conventional neck gaiters are used and have primarily one element for function, or one function, which is for warmth in cold environments, whether they are for sports, any outdoor activity, or for working in the outdoors. Manufacturers' neck gaiters typically cover some portion or all of the neck, and have an open portal for pulling the gaiter over the head to position around the neck using “wicking” coupled with warmth material to keep sweat away and keep the human body/skin dry and warm. They are designed for cold weather without bulk using a double-sided fabric that “wicks” the moisture away from the skin and circulates body heat.

Other neck items on the market relate to neck braces, as used in post trauma scenarios to stabilize the neck, or neck braces designed for motocross to further protect the neck, or in NASCAR using the HANS System to also protect the neck, and assist to stabilize the head in an accident—these are two examples.

### SUMMARY OF THE PREFERRED EMBODIMENTS

In accordance with a first aspect of the present invention there is provided a neck gaiter system configured to be received on the neck of a wearer that includes a gaiter member that includes a neck portion and a clavicle portion, a sensor system or sensor arrangement system that includes at least one gyro sensor and/or at least one impact crash force sensor positioned on the gaiter member, an actuation system in electrical communication with the sensor system, and a bladder positioned on at least the neck portion of the gaiter member. The bladder is in fluid communication with the actuation system. When the sensor system senses a predetermined rotational change (or linear or directional change), the actuation system inflates the bladder. In a preferred embodiment, the sensor system includes an accelerometer. In another embodiment, the sensor system can be configured such that when it senses multiple predetermined changes or events the bladder is inflated. For example, the sensor system can be configured such that when it senses two or more of a predetermined rotational change, a predetermined velocity (linear and/or angular) change and/or a predetermined directional change, the actuation system inflates the bladder. The sensor system can also include at least one velocity sensor, at least one impact sensor, as well as the gyro sensor cycling over 800 times per second and at least one Bluetooth sensor.

In a preferred embodiment, the sensor system includes a body sensor in communication with the gyro sensor, impact sensor and/or biometric sensor. The sensor system can include other sensors (or the sensors can all be included in a single unit) that sense linear acceleration changes (either an increase or decrease in acceleration or velocity, such as being thrown from a horse, motorcycle, ATV or a direct impact), directional changes, velocity changes, a forceful impact or biometric changes. For example, the system can include a biometric sensor(s) that monitors or senses heart rate (resting heart rate, current heart rate, hear rate variability), blood pressure, blood oxygenation level, step count, EKG. The neck gaiter system can also include GPS capability with real-time data transmission (e.g., via Bluetooth) through software and available to doctors and an app on a mobile device, tablet, or other, and the ability to call over cellular, satellite or other network should the wearer desire. The sensor system with a single type of sensors or multiple sensors working together is configured to determine or sense predetermined angular changes, velocity changes, directional changes and/or linear changes that signal an inflation event. This is communicated to the actuation system and the bladder is inflated to protect the wearer. In a preferred embodiment, the neck portion of the bladder is ring shaped. In particular, the neck portion can include a ring just under the jaw or mandible of the wearer where the bladder is ring shaped.

Preferably, the sensor system is switched from a deactivated mode to an activated mode when the body sensor senses a predetermined factor indicating that the neck gaiter system being worn by the wearer is reached and the airbag is deployed. The factor can be contact with the user's skin or other sensing factor. In a preferred embodiment, the body sensor is positioned to contact the wearer's skin near the carotid artery or other artery available in the neck and clavicle area.

The method embodies an inherent bladder airbag system that is deployed with resulting over rotation on the digital gyroscope sensor arrangement, and/or detrimental impact on the impact crash sensor arrangement using an actuation mechanism and an inflation interconnected bladder system, or interconnected tube(s) resulting in release of the inert compressed inert gas in a cartridge actuation mechanism using a triggering mechanism, which is held in an interior pocket of the neck gaiter resulting in aspiration of the inflatable safety bladder protection collar or gaiter. The invention also includes in the safety detached neck collar/gator protection system breathable double knit fabric gaiter worn over the neck, a sensor arrangement in the form of a digital gyroscope arranged with specific characteristics surrounding the wearers positioning, a single impact lead sensor (more than one lead can be used), an actuation mechanism and an inflation tube, a triggering mechanism, an inert compressed gas cartridge (a non-inert gas can be used), a sensor to engage the digital gyroscope, and an inflatable safety bladder system. In another embodiment the system can communicate via Bluetooth. The sensor system can communicate with the actuation system via a wired or a wireless (e.g., Bluetooth) connection.

The method also embodies an inherent bladder airbag system that is deployed with resulting change in the wearers positioning from perpendicular to the ground to other compromising positioning or attitude change going past a safe range of vertical to forty five degrees and greater where there is a distinct possibility of landing on your head and neck in a dangerous and compromising position, dependent upon the use or sport activity the user is engaged, or extension or hyper-extension or flexion or hyper-flexion resultant from head and resulting neck impact through a digital gyroscope sensing device resulting in release of an inert compressed gas cartridge actuation mechanism held in an interior pocket of the gaiter resulting in aspiration of the inflatable safety bladder protection collar.

The method also embodies an optional single impact lead system that may be simply attached to the helmet of the wearer, which embodies an inherent bladder airbag system that is deployed with resulting impact force as applied to the helmet at point of impact, or compression, and the resultant transfer of impact force to the sensor and through the sensor lead attached to the helmet transmitted to the actuation mechanism activating the inert compressed inert gas in a cartridge held in an interior pocket of the neck gaiter resulting in aspiration of the inflatable safety bladder protection collar. In another embodiment the system can communicate via Bluetooth.

The system of the present invention may contain both the digital gyroscope, as well as the sensor lead in release of an inert compressed gas cartridge actuation mechanism held in an interior pocket of the gaiter resulting in aspiration of the inflatable safety bladder protection neck gaiter. The system may also include sensors to engage the digital gyroscope, so the digital gyroscope recognizes when it is not being worn—the digital gyroscope will activate once it is on the wearer's body, and will disengage if not deployed once taken off the wearer's body.

The invention generally relates to safety neckwear, such as for activities and sports including, but not limited to football, soccer, hockey, lacrosse, motorcycling, ATV, motocross, mountain biking, bicycle cross, all equestrian forms, as well as for health and safety cervical protection used post accidents for the neck in the forms of cervical collars, traction collars, first response collars, cervical othosis, low density packs, and foam collars, and further includes, other sports using some form of neck warmth. The invention also generally relates to protective gear, such as padding, etc.

Various activities, in both contact and non-contact sports, and as examples: football, soccer, hockey, lacrosse, motorcycling, motocross, mountain biking, bicycle cross, all equestrian forms, do not always require, but typically use helmets, yet the neck area remains unprotected for incurring concussive forces relating to impacts with other players helmets, other players padding throughout the body, including impacts with elbows, knees, etc., as well as impacts with all forms of equipment, animals, the ground, and so forth. Recent attempts to further protect participants from impact injuries predominantly surrounds the head, and little attention is paid to participants necks, and the severe impacts forces that may be sustained during such activities.

Various activities sustain impacts with other players, and other activities sustain impacts with objects, and in a lot of the cases some of the incidents are anticipated, and some are not, as a player/participant has to be able to see the incoming impact, as well as have the time to be able to react, meaning reacting with some sort of movement away from the incident, or the ability to position the players body in order to improve the impact, or have the time to “tense” the body and neck area. By tensing the neck area the player improves the overall ability of the body, and more specifically the head and neck to sustain the impact, by the “mass of the head is essentially the mass of the body. In a relaxed state, the mass of the head is essentially only its own weight and therefore the same degree of force can impart far greater acceleration.” The neck takes a great deal of force, but in the relaxed state it is just as vulnerable as an “independent” yet connected piece of the anatomy reacting to force, acceleration, rotational acceleration and velocity, as well as flexion and extension.

Additionally, the neck is an important aspect of head injury, as whiplash indirectly affects the head with sufficient force applied. Concussions may occur in this instance from neck whiplash, and it is exponentially increased in players who sustained previous concussions. Injuries to the head and neck are the most frequent catastrophic sports injuries.

Various improvements over the recent years have been made to the assorted helmets, however, in general, the impact dissipation and energy attenuation claims are not meeting their design specifications, as touted by the manufacturers, and necks remain open and vulnerable, and especially in children. Since a player wears a helmet for a considerable period of time during practices and games, it would be desirable to also protect the neck, as noted above the neck is an important aspect of protecting the head and brain from more extensive damage. It would be desirable if the neck has protection in addition to the head to assist integrating the neck and head as one strong “unit,” thereby improving the opportunity for the player to sustain an impact at the most optimal body engagement to address an impact.

The developing brain may indeed be more prone to injury than an adult, mature brain because neurons are growing faster and connections are still being made. There is not the redundancy that there is in an adult brain.” In addition, “younger children have proportionately bigger heads than adults, and weak necks, so the ability to sustain a blow to the head is not as great. A blow of the same degree exerts more acceleration force to a child (particularly under the age of 12 or so) than to an adult, because of the relative weakness of the neck musculature, which essentially acts as a shock absorber. Diffuse cerebral swelling is another serious condition that may be found in the child or adolescent athlete. This may also explain why female athletes seem to be more susceptible to concussive injuries than men, whose necks tend to be stronger. Also, unlike NFL and college players, many children have not yet learned how to tackle and protect themselves. Additionally, children's brains take longer to recover, and concussive symptoms do not show up immediately or within a couple of months like adults, and actually may show up years down the road post trauma.

While it is the desire and goal that a helmet, and other types of protective neck devices to prevent injuries from occurring, it should be noted that as to the protective neck gaiter of the present invention, due to the nature of sports in general, no protective equipment, can completely, totally prevent short term or long term injuries to those individuals playing. It should be further noted that no protective helmet, neck protection and other equipment can completely prevent injuries to a player, and as an example, if the player uses their head in an improper or illegal manner, such as to butt, ram, or spear an opposing player, which is in violation of most sports regulations. Improper use of a players head to butt, ram, or spear an opposing player can result in severe brain, head and/or neck injuries, paralysis, or death to the sports player, and may further include the possible injury to the sports player's opponent. No protective neck gaiter, or other protective neck gaiter(s), including other transportable safety equipment, such as that of the current invention, can completely prevent head, brain, chin, spine, or neck injuries a sports player might receive while participating in such contact or non-contact sports. The protective neck gaiter of the present invention, and other transportable safety equipment as stated herein are believed to offer improved protection, but it is believed that no protective neck gaiter can, or will ever, totally and completely prevent neck injuries to sports players.

Sports for decades has been traditionally played without any protective neckwear. Certain sports, such as motocross have neck braces that are being used, but football, lacrosse, hockey, and so on do not use any neck protection.

### BRIEF DESCRIPTION OF THE DRAWINGS

The invention may be more readily understood by referring to the accompanying drawings in which:

FIG. 1 illustrates a neck gaiter system of the inner layer of the double wicking layer of the disposable advanced sports safety neck gaiter technology system, and shows placement on the user or wearer's neck. FIG. 1 also illustrates the bladder air bag system, and the inert gas canister, and the double wicking material for both the inner and outer layers.

FIG. 2 schematically illustrates interior wicking layers of material view with double wicking material of an example of a neck gaiter system, with a bladder air bag system and an inert gas canister with a single lead from a crash sensor, as well as a Bluetooth sensor system.

FIG. 3 schematically illustrates a bladder air bag system, with an inert gas canister contained within a pocket in the double wicking material, and a digital gyroscope contained within a pocket in the double wicking material, as well as a Bluetooth sensor system.

FIG. 4 schematically illustrates a bladder air bag system, with an inert gas canister contained within a pocket in the double wicking material with a single lead, and a digital gyroscope contained within a pocket in the double wicking material.

FIG. 5 schematically illustrates a digital gyroscope to be specifically placed in the pocket of the neck gaiter on a user or wearer.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments.

Reference in this specification to “one embodiment” or “an embodiment” means that a particular feature, structure, or characteristic described in connection with the embodiment is included in at least one embodiment of the-disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

An articulating body protective device for protection from impact-based injuries, especially relating to sports activities, is provided by a fabric outer neck gaiter element or garment and pound force impact force related elements, which are strategically provided in predetermined locations within the layers of the neck gaiter outer garment for protecting the human neck and prospectively assisting the head from injuries related to impact during sports or athletic activities.

The cartridge actuation mechanism includes a triggering device that can be actuated to open the cartridge by means of a chemical charge in the cartridge inflator actuation system. The inflator sets off a chemical charge, producing an explosion of compressed inert gas, filling up the bladder airbag system. The actuation is in response to either a resulting change in the wearers positioning from perpendicular to the ground to other compromising positioning or attitude change going past a safe range of vertical to forty five degrees and greater where there is a distinct possibility of landing on your head and neck in a dangerous and compromising position, or a compressive force of predetermined impact force or magnitude, and in doing so the inflator sets off the inert compressed gas or chemical charge. A compressed gas cartridge is held in an interior pocket of the neck gaiter system, specifically in the clavicle member of the neck gaiter system, and attached to an inflatable bladder airbag system. The compressed inert gas cartridge connects to the bladder airbag system through a cartridge actuation mechanism. The actuation mechanism is resultant of exceeding a specified degree preset or predetermined in the digital gyroscope, or pound force of compressive pressure from impact force or velocity changes.

The digital gyroscope system embodies the ability to sense the wearer's positioning relative to the ground with a pre-set specified degree of angle or the impact sensor lead detects pound force respectively and velocity changes whether it is resulting from hyper-flexion, or hyper-extension, or linear acceleration, or g-force, or acceleration, or angular acceleration, or rotational acceleration. This digital gyroscope and impact sensor system embodies the ability to sense the wearer's positioning relative to the ground with a pre-set specified degree of angle thereby sending a signal to the actuation mechanism triggering the compressed inert gas cartridge to release the inert gas, which immediately and instantly inflates the bladder airbag system thereby protecting the neck and clavicle of the user or wearer. Also, this impact sensor system embodies the ability to sense pound force and velocity changes thereby sending a signal to the actuation mechanism triggering the compressed inert gas cartridge to release the inert gas, which immediately and instantly inflates the bladder airbag system thereby protecting the neck and clavicle of the user or wearer. The digital gyroscope senses body positioning relative to the ground, so any change in positioning past the pre-set degree of angle relative to the ground will inflate the inert gas (argon is one example) canister and bladder system. The impact force may be sensed from “contact” whether it is from a “contact surface” including, but not limited to: helmets worn by human wearers, contact with other humans not wearing helmets, and impact with other parts of the human body, such as, but not limited to: legs, shoulders, elbows, hands, wrists, knees, feet, ankles, shoes worn by the human, hips, and other body parts and the like, plus the impact force may be sensed from “contact” whether it is from a “contact surface” such as, but not limited to: the ground consisting of grass, dirt, man-made turf, such as astro turf, or synthetic turf used instead of grass, snow, ice, asphalt, clay, concrete, other surfaces and the like. The impact sensor also detects “senses” velocity changes where upon the system is activated to protect the wearer. The digital gyroscope system will engage when worn by the wearer.

The neck gaiter member, in some instances, will exhibit a layered configuration that includes a soft comfortable double layer of wicking material, a strong, pliable and durable bladder system (formed out of some polymer, such as flexible plastic, including, but not limited to thermoplastics including polyethylene, polypropylene, polystyrene, polyvinyl chloride, and polytetrafluoroethylene), a digital gyroscope, and a Bluetooth system or a lead system attached and originating from the electrode impact sensor with the lead the wearer attaches to the exterior of their helmet, and the lead goes back to the actuation mechanism thereby triggering the release of the inert gas contained in the compressed inert gas cartridge that connects to the bladder airbag system through a cartridge actuation mechanism, with the inert gas release inflating the bladder airbag system, which is the resultant of either changes sensed by the digital gyroscope system, or pound force of compressive pressure from impact force to the human head, or from velocity changes. Suitable flexible yet resilient plastics used for the bladder airbag system using polyethylene, polypropylene, polystyrene, polyvinyl chloride, and polytetrafluoroethylene that is flexible, but durable for holding shape under impact conditions, excessive heat conditions, not losing its strength or shape. The inert gas cartridge system may have a cylinder shape and consist of, but not be limited to metals, plastics, screws, nozzles, seals, clamps, and other components, parts and materials not listed. The bladder airbag system, digital gyroscope, digital gyroscope activation sensing mechanism, electronic sensor system, lead, inert compressed gas cartridge, neck gaiter system may be joined to one another in various different methods, such as mechanical connectors, stitching or sewing, adhesives, cements, glues, fusing techniques, and other materials or techniques not listed.

The current invention is a disposable advanced sports safety neck gaiter technology system and apparatus that is designed to protect the neck from injury resulting from sports and other impacts. The current invention is designed to protect the neck from the impact force. The current invention, as a disposable protective sports safety neck gaiter, includes inside the protective neck gaiter in a variety of form(s), such as “Bluetooth,” “leads,” “inert argon or other gas canister” “sensors,” “triggering, or actuation mechanism,” “aspirated,” “bladder system,” “disposable,” and/or “compressed” or in any combination or layers as needed in the protective neck gaiter contained within the neck gaiter having an outer surface, an inner surface, a front region, a rear region, and two side regions, and with the neck gaiter bladder system from the previous filed provisional patent already noted inside the protective neck gaiter in a variety of combination or layer or layer(s) contained within the neck gaiter having an outer surface, an inner surface, a front region, a rear region, two side regions, and top and bottom apertures.

The current invention further comprises an improved reinforcement system for the neck for use in a variety of advanced sports safety neck gaiters. In one embodiment, encompasses a digital gyroscope and sensor system designed to address uncontrollable ejection from a vehicle, bike, ATV, animal, or simply falling past the degree or angel where the player or users neck and head may be compromised, as stated herein and articulated so that the resultant impact results in a force component engaging the advanced sports safety neck gaiter. The aspirated neck safety gaiter device(s) can be incorporated into a wide variety of advanced sports safety neck gaiters of a variety of sizes and configurations to produce impact protection and/or other advantages.

The disposable aspirated/inflatable safety detached neck collar/gaiter protection system of the previous invention referenced herein, and of the current invention is designed to attempt to protect a wearer of the protective neck gaiter from neck injury of the present invention. “The present invention consists of methods and apparatus, which can inexpensively and efficiently manufacture neck gaiter(s) in high volume with a specifiable range of design of reinforcement(s) having neck protection as needed. Previously unavailable neck protection may assist in overall safety performances for safety helmets and neck gaiter/gear protection in general.

The following synopsis represents a summary of aspects of the invention to provide a basic understanding of the invention, and the purpose of the invention. This summary is just that to provide an overview of the invention, and is not intended to identify all key critical elements of the invention, or to define/describe the scope, capacity or opportunity of the invention. The summary simply provides some concepts of the invention in a general form, as an introduction to the comprehensive description outlined below.

Aspects of the invention pertain to neck members and neck receiving devices, such as a neck gaiter system (i.e. neck gaiters already manufactured principally to provide warmth to the neck of the user for athletics, and any other daily use where protection from the weather is required), which includes a neck member, which is engaged as one complete unit, engaged as a double layered moisture wicking system. The neck member may include a digital gyroscope, and/or an electronic sensor system with Bluetooth or lead(s) or wire(s) leading to the actuation system, which may include or define a cavity or void to house or hold an inflatable bladder system as a neck receiving device. The neck member may also include a an impact or velocity change sensing mechanism or sensor system correlated to the digital gyroscope, which activates the digital gyroscope when worn by the wearer. The neck member may also include or define a cavity or void to house or hold an inflatable bladder system as a neck receiving device. The neck member, at the base of the neck or at the clavicle, may also include or define a cavity(s), void(s) or pocket(s) receiving device to house or hold a triggering device and a cartridge inflator actuation system, and a digital gyroscope.

Additional aspects of the invention relate to sample methods for providing neck protection using a cartridge actuation mechanism includes a triggering device that can be actuated to open the cartridge by means of a chemical charge in the cartridge inflator actuation system, filling up the bladder airbag system.

Such methods may include athletic vests, auto airbags, as receiving members.

Aspects of the invention pertain to neck member (i.e. neck gaiters already manufactured principally to either provide warmth to the neck, and/or wick moisture away from the wearer for users for athletics, and any other daily use where protection from the weather is required), which includes a neck member, as a single unit, engaged as a double layered moisture wicking system. The neck member may include an enclosed plastic or flexible polymer system with a cavity to receive the inflatable inert gas. The neck gaiter may also include or define a cavity, void or pocket receiving device to house or hold the inert gas canister and actuation mechanism.

In accordance with the present invention embodiment, the apparatus comprises neck protection in the form of a neck gaiter, and the neck gaiter comprises a multi-layered sidewall, front-wall, and back-wall, and defines an opening(s) for a head of a wearer. The multi-layered wall(s) of the neck gaiter comprise an outer fabric layer, and an inner fabric layer positioned proximate to the outer fabric layer. The neck gaiter comprises a multi-layered sidewall, front-wall, and back-wall or panel(s). The multi-layered sidewall extends from multi-layered top wall or panel as one contiguous unit, and defines an opening(s) for a head of a user or wearer. The neck gaiter is stretchable between a relaxed relationship and an expanded relationship when pulled over the head of the wearer, and then placed upon the neck and clavicle of a wearer.

In accordance with another embodiment, the apparatus comprises a neck gaiter having a longitudinal axis. The neck gaiter also comprises a sidewall extending circumferentially about the longitudinal axis. The sidewall comprises an outer wicking stretchable fabric layer, an inner fabric layer, and in between the outer and inner fabric layer is a closed/sealed tube made of polyurethanes, polyolefins or any polymeric material attached to both the outer and inner stretchable fabric layer(s), where the two layers define a cavity, void or pocket receiving device to house or hold the inert gas when released through the actuation mechanism system. The inner layer also comprises a layer positioned within the outer layer, the inner layer having a full circumference surface, where it is circumferentially spaced in relationship to the outer layer. The inner layer has a top edge surface, a bottom edge surface and a circumference sidewall surface to define a cavity, void, pocket there between the inner layer and the outer layer.

In accordance with another embodiment, the apparatus comprises a neck gaiter having an outer layer that has a top edge surface, a bottom edge surface and a circumference sidewall surface to define an additional small cavity, void, pocket to house or hold the inert argon canister and actuation mechanism, as well as an additional small cavity, void, pocket to house or hold the digital gyroscope, and a sensor system next to the wearers neck.

In accordance with yet another embodiment, the apparatus is to be worn by a user independent of a hard surface helmet shell, and the current invention comprises stand-alone neck protection in the form of a neck gaiter to be worn by the users or wearer when participating in sports. The neck gaiter comprises a wicking stretchable outer fabric layer, an inner fabric layer, a space in between the inner and outer layers where there is a definition of a cavity, void or pocket receiving device to house expended gas contained within the inert canister until triggered by the actuation mechanism. The stretchable fabric layer(s) are configured to contact and generally conform to the neck of a wearer.

The invention generally relates to safety neck systems, such as for activities and sports including, but not limited to football, hockey, riding, ATV, motocross, bicycling, and further includes, other contact and non-contact sports requiring some form of neck and head protection. This invention relates generally to a disposable neck protection system designed to protect the human neck from injury resulting from sports accidents and impacts. Contact and non-contact sports that both suffer from neck issues, as well as from concussions, in which acceleration, angular, rotational, linear with possible extension or flexion motion resulting from an impact force that calls for or requires protection for both the neck and the head.

The method of the present invention embodies an inherent system contained in a breathable wicking material, such as sold by Under Armour and others with an outer fabric layer and an inner fabric layer, or closed/sealed tube made of polyurethanes, polyolefins or any polymeric material to receive the inert gas once triggered by the actuation mechanism locked within the outer fabric layer and the inner fabric layer in a permanently enclosed environment. The present invention also includes and contained within the outer fabric layer a pocket to contain the inert argon, and other in use today inert gases, gas canister and actuation mechanism, similar to that taught in U.S. Pat. No. 9,986,777, the entirety of which is incorporated by reference herein. The present invention also includes in the neck gaiter system breathable double fabric knit as the exterior/outer fabric layer and the inner fabric layer that also may include a lead system, and may include a Bluetooth system that is also embedded to work with the actuation mechanism. The present invention is a disposable advanced sports safety neck gaiter technology system and apparatus that is designed to protect the neck from injury resulting from sports and other impacts using an outer layer of wicking material, with an inner layer and sealed in between the two fabric layers is a bladder system using a polyurethanes, polyolefins or any polymeric material to receive the dispersion of the inert argon gas contained within the cylinder in a disposable advanced sports safety neck gaiter technology system.

The method embodies a disposable advanced sports safety neck gaiter technology system when worn by the wearer and activated that is set in motion either by surpassing the pre-set limits of degree change by a digital gyroscope, or by the impact force or velocity changes on the Bluetooth or lead attached to the helmet shell that is designed to actuate or trigger the mechanism to release the gas into the bladder system contained within the two fabric layers of the neck gaiter when a pre-set amount of impact force is applied to the wearer whether or not they are wearing a helmet shell.

The present invention looks at a neck protective device for protection from impact-based accidents and injuries, especially relating to sports activities, is provided by a fabric or wicking outer and inner fabric layer(s) material/element/member, or garment, with an inner enclosed plastic or flexible polymer system with a cavity to receive the release of the inert gas contained within the canister housed in a pocket in the neck gaiter located at the base of the back of the neck above the clavicle.

The present invention's technology and its ability to protect the neck from accident or sports-related impact force whether it is resulting from linear acceleration, angular acceleration, rotational acceleration, hyper-flexion, or hyper-extension at the point of impact will apply to, but not be limited to the neck and heads contact with other humans wearing neck gaiter/helmets, and impact with other parts of the human body, such as, but not limited to: legs, shoulders, elbows, hands, wrists, knees, feet, ankles, shoes worn by the human, hips, and other body parts and the like, plus the impact force may be sensed from “contact” whether it is from a “contact surface” such as, but not limited to: the ground consisting of grass, dirt, man-made turf, such as astro turf, or synthetic turf used instead of grass, snow, ice, asphalt, clay, concrete, other surfaces and the like.

The present invention will have an outer layer attached to the inner layer via at the top and bottom of the neck gaiter, which may be constructed using stitching or sewing, sealants or adhesives, heat sealing, cements, glues, fusing techniques, and other materials or techniques not listed, or a combination thererof, and the inner bladder system contained and sealed therein and contained within the inner layer may be constructed using stitching or sewing, sealants or adhesives, heat sealing, cements, glues, fusing techniques, and other materials or techniques not listed, or a combination thererof. The exterior or outer fabric layer, the inner fabric layer, the inner durable plastic bladder layer, lead(s), sensor(s), triggering/actuation mechanism, inert argon gas canister, may be joined to one another in various different methods, such as mechanical connectors, stitching or sewing, adhesives, cements, glues, fusing techniques, and other materials or techniques not listed.

The following description includes various examples of the invention, which are referenced and reference is made to the associated drawings, which form a part hereof, and in which are shown by way of illustration example systems. Also, the following description includes various examples of the invention, which are referenced and reference is made to the associated drawings, which form a part hereof, and in which are shown by way of illustration example environments and usage the invention may be employed. It is to be stated that other configurations, usages, aspects of use, parts, portions, example systems may be used and structural and functional modifications or alterations may be made without taking leave from the scope of the present invention. Terms, such as “around,” “through,” “top,” “bottom,” “side,” “above,” “below,” “underneath,” “over,” “clear,” “transparent,” “inner,” “outer,” “coils,” “fluids,” “soft,” “wicking,” “single,” “double,” “force,” “impact,” “linear,” “rotational,” “angular,” “acceleration,” etc. may be used to describe the invention, and the various examples, and example aspects, facets, features, elements of the invention, these terms are used herein as a matter of descriptors and for practicality and expediency based upon the example orientations as shown in the illustrations. Nothing in this specification should be construed as requiring a specific three-dimensional orientation of structures in order to fall within the scope of this invention.

An aspirated inflatable safety protection neck gaiter system and apparatus designed to protect the human neck from injury resulting from sports accidents or sports impacts with either a digital gyroscope, and/or a crash sensor arrangement in which acceleration, angular, rotational, extension or flexion/extension motion resulting from an impact force requires deployment of an apparatus protection device. The method embodies an inherent bladder airbag system that is deployed with resulting impact force either through the pre-set degrees on the digital gyroscope, and/or the sensor arrangement using an actuation mechanism and an inflation interconnected bladder system, or tube(s) resulting in release of the inert compressed inert gas in a cartridge actuation mechanism using a triggering mechanism, which is held in an interior pocket of the neck gaiter resulting in aspiration of the inflatable safety bladder protection neck gaiter.

A disposable advanced sports safety neck gaiter technology system/member and apparatus designed to protect the human neck from injury resulting from sports accidents and sports impacts in which linear acceleration, angular, and/or rotational acceleration, deceleration or velocity resulting from an impact force, as well as hyper-flexion or hyper-extension requires deployment of an apparatus protection device at impact. The following also outlines: A disposable advanced sports safety neck gaiter technology system/member and apparatus designed to protect the human neck from injury resulting from sports accidents and sports impacts in which linear acceleration, angular, and/or rotational acceleration, deceleration or velocity resulting from an impact force, as well as hyper-flexion or hyper-extension requires deployment of an apparatus protection device at when the wearer goes past a pre-set degree or angle that is past 45 degrees of perpendicular to the ground, or what is set in a digital gyroscope contained within the neck gaiter.

General Description of the advanced sports safety technology neck gaiter system receiving devices according to the invention.

Some aspects of the present invention relate generally to neck gaiter systems, to airbag protection devices, to protective equipment for safety in sports, and other uses. The advanced neck gaiter protection technology system fits on a wearer's neck. The electronic sensor system may occupy one or more placements within the base of the neck portion of the neck gaiter system. The digital gyroscope may occupy one placement within the neck gaiter. The single electronic lead or wire may occupy one or more placements within the neck or clavicle area portion of the neck gaiter system. The Bluetooth may occupy one placement within the neck or clavicle area portion of the neck gaiter system. The inflatable safety bladder airbag system may occupy one or more placements within the neck portion of the neck gaiter system. The inert compressed gas canister actuation mechanism may occupy one or more placements within the neck and clavicle portion of the neck gaiter system. Any of the sensors discussed herein may be placed in one or more areas of the neck portion of the neck gaiter system.

The neck gaiter system may be formed from a variety of comfortable wicking materials already in use in today's market, and may be formed with a variety of characteristics in the prior art. The single electronic sensor arrangement system may be formed from a variety of materials already in use in today's market, and may be formed with a variety of characteristics. The inherent bladder airbag system may be formed from a variety of materials already in use in today's market, and may be formed with a variety of characteristics. The actuation mechanism may be formed from a variety of materials already in use in today's market, and may be formed with a variety of characteristics. The digital gyroscope mechanism may be formed from a variety of materials already in use in today's market, and may be formed with a variety of characteristics. The cartridge may be formed from a variety of materials already in use in today's market, and may be formed with a variety of characteristics. The inert compressed gas may be formed from a variety of materials already in use in today's market, and may be formed with a variety of characteristics. The triggering mechanism may be formed from a variety of materials already in use in today's market, and may be formed with a variety of characteristics.

Example Advanced Neck Gaiter Technology Systems According to the Invention. Aspects of the invention relate to safety systems in sports and relates to an aspirated inflatable safety neck gaiter system, or an aspirated inflatable safety protection system means any device that a user places on or over some portion of the human body. The neck gaiter safety system receiving device, (i.e. a neck gaiter system designed to protect the users neck area), which is an aspirated inflatable safety protection neck gaiter system and apparatus including a neck gaiter with an inner layer and an outer layer, the inner and outer layers including at least a neck member, and a clavicle member, at least one member of the neck member including one or more pockets in the neck portion of the article of the neck gaiter system.

The neck gaiter member portion of the neck gaiter contains the electronic sensor arrangement system, including at least one electronic sensor, at least one digital gyroscope, and at least one sensor distributed at the base of the neck portion of the gaiter system and are stitched, or some form of contact to maintain positioning in between the layers of materials of the neck gaiter, but not being limited and may touch the wearers skin on the neck. The digital gyroscope is also located at the base of the neck portion of the neck gaiter system. The electronic sensor is located at the base of the neck member portion of the neck gaiter system, or at the side member of the neck gaiter system. Additionally, the neck member may include one lead or wire extending therefrom the electronic sensor arrangement system. Additionally, the neck member may include a wireless Bluetooth sensor system. When the digital gyroscope, single (or multiple) electronic sensor system, coupled with one single lead or wire (or Bluetooth capability) is inserted, or attached with the best method of attachment, in the cavity between the two layers of wicking material in the neck gaiter member, the protrusions may extend vertically and laterally and/or upward or downward between the two material layers (i.e. the digital gyroscope, impact and other sensors will be as flat as possible, but will have some minor protrusions). The wire or lead will be also located in the cavity between the layers of wicking material in the neck gaiter and neck members, the protrusions may extend vertically and laterally and/or upward or downward between the two material layers (i.e. the lead will be as flat as possible, but will have some minor protrusions). The Bluetooth and sensor system may also be touching the wearers skin. The bladder airbag system will be also located in the cavity between the layers of wicking material in the neck gaiter members, the protrusions may extend vertically and laterally and/or upward or downward between the two material layers (i.e. the bladder airbag system will be as flat as possible, but will have some minor protrusions). The cartridge actuation mechanism will be also located in the cavity between the layers of wicking material in the neck gaiter into the base of the neck or clavicle members, the protrusions may extend vertically and laterally and/or upward or downward between the two material layers (i.e. the cartridge actuation mechanism will be as oval, or round, or flat, and as small as possible, but will have some protrusions).

The electronic sensor actuation system of an example embodiment of the invention may occupy a minor portion of the neck members of the neck gaiter system, i.e., less than 10%, at least 5%, of the neck member of the neck gaiter system and region or area. The digital gyroscope system of an example embodiment of the invention may occupy a minor portion of the entire neck and clavicle members of the neck gaiter system, i.e., at least 10%, or at least 5%, of the neck and clavicle member of the neck gaiter system and region or area. The electronic lead or wire system of an example embodiment of the invention may occupy a minor portion of the entire neck and clavicle members of the neck gaiter system, i.e., at least 5%, or at least 1%, of the neck and clavicle member of the neck gaiter system and region or area. The bladder airbag system of an example embodiment of the invention may occupy a significant portion of the entire neck and clavicle members of the neck gaiter system, i.e., at least 50%, or at least 60%, or at least 70%, or at least 80%, or even at least 90% of the neck and clavicle member of the neck gaiter system and region or area. The cartridge actuation mechanism system of an example embodiment of the invention may occupy a significant portion of the neck and clavicle members of the neck gaiter system, i.e., at least 10%, or at least 5%, of the neck and clavicle member of the neck gaiter system and region or area. The cartridge actuation mechanism may also further include a trigger mechanism connecting the leads to the cartridge containing the inert compressed gas. The electronic sensor actuation system may also further include connections to a single lead or wire, a digital gyroscope, or a Bluetooth and sensor system. The electronic sensor actuation system may also further include a method to attach the electronic sensor to the material layers, and which may reside in between the two layers of wicking material of the neck gaiter system. The cartridge may also have a method of attaching or connecting beyond being located in a pre-designed, sewn, or constructed pocket to hold the cartridge. The digital gyroscope may also have a method of attaching or connecting beyond being located in a pre-designed, sewn, or constructed pocket to hold the cartridge. The cartridge when inserted in the cavity or void designed in the neck gaiter system, specifically in the clavicle member of the neck gaiter system, may have one or more protrusions that may extend laterally and vertically, may have depth, may have height, and may be in three dimensional form.

As already described, the one or more protrusions included in the neck member and the clavicle member of the neck gaiter system as an example of the embodiment of the invention, all of which may extend laterally, vertically, may have depth, may have height, and may be in three dimensional form.

Also as noted, the one or more protrusions included in the neck member, as an example of the embodiment of the invention, may include a single electronic sensor oriented in between the two layers of wicking material of the neck gaiter system, while not visible will be noted due to the protrusions extending vertically, horizontally, having depth, having height and being in three dimensional form.

Also as noted, the one or more protrusions included in the neck member, as an example of the embodiment of the invention, may include a Bluetooth sensor system located next to the wearers skin, or a single wire or lead oriented in between the two layers of wicking material of the neck gaiter system, while not visible will be noted due to the protrusions extending vertically, horizontally, having depth, having height and being in three dimensional form.

Also as noted, the one or more protrusions included in the neck member, as an example of the embodiment of the invention, may include a digital gyroscope oriented in between the two layers of wicking material of the neck gaiter system, while not visible will be noted due to the protrusions extending vertically, horizontally, having depth, having height and being in three dimensional form.

Also as noted, the one or more protrusions included in the neck member, as an example of the embodiment of the invention, may include sensors oriented in between the two layers of wicking material of the neck gaiter system, while not visible will be noted due to the protrusions extending vertically, horizontally, having depth, having height and being in three dimensional form.

Also as noted, the one or more protrusions included in the neck member and the clavicle member, as an example of the embodiment of the invention, may include one or more bladder airbag systems oriented in between the two layers of wicking material of the neck gaiter system, while not visible will be noted due to the protrusions extending vertically, horizontally, having depth, having height and being in three dimensional form.

Also as noted, the one or more protrusions included in the clavicle member, as an example of the embodiment of the invention, may include one or more cartridge actuation mechanism oriented in between the two layers of wicking material of the neck gaiter system, while not visible will be noted due to the protrusions extending vertically, horizontally, having depth, having height and being in three dimensional form.

The bladder airbag system may be formed of a variety of materials and/or include a variety of features or element to alter or adjust characteristics of the bladder airbag receiving device. For example, the pliable and durable bladder system may be formed out of some polymer, such as flexible plastic, including, but not limited to thermoplastics including polyethylene, polypropylene, polystyrene, polyvinyl chloride, and polytetrafluoroethylene.

The electronic sensor system may be formed of a variety of materials and/or include a variety of features or element to alter or adjust characteristics of the electronic sensor system receiving device.

The cartridge actuation system may be formed of a variety of materials and/or include a variety of features or elements to alter or adjust characteristics of the cartridge actuation system. This may be an off-the shelf cartridge actuation system utilized in other products.

The digital gyroscope actuation system may be formed of a variety of materials and/or include a variety of features or elements to alter or adjust characteristics of the cartridge actuation system. This may be an off-the shelf cartridge actuation system utilized in other products.

The sensor system may be formed of a variety of materials and/or include a variety of features or elements to alter or adjust characteristics of the cartridge actuation system. This may be an off-the shelf cartridge actuation system utilized in other products.

Example Methods of Providing and Using Aspirated Inflatable Safety Neck Gaiter Receiving Device Systems According to Examples of the Invention. Additional aspects of the invention include methods of providing and methods of using neck gaiter systems.

For example, to insert the cartridge inflator actuation mechanism system, wherein the cartridge contains compressed inert gas, and wherein the cartridge contains a trigger mechanism; to insert the digital gyroscope or impact crash electronic sensor arrangement including leads distributed/situated throughout the neck gaiter member; the neck member including one or more apertures; to insert the bladder airbag system at least a portion of the neck gaiter system may be removed or separated between the two wicking layers of material where each of the noted component parts above will be inserted.

To insert the cartridge inflator actuation mechanism system, wherein the cartridge contains compressed inert gas, and wherein the cartridge contains a trigger mechanism into the neck gaiter system, at least a portion of the clavicle member of the neck gaiter system, may be removed or separated between the two wicking layers of material where each of the noted component parts above will be inserted.

To insert the impact crash electronic sensor arrangement including a single lead distributed/situated at the base or clavicle neck member including one or more apertures into the neck gaiter system, at least a portion of the neck and clavicle members of the neck gaiter system, may be removed or separated between the two wicking layers of material where each of the noted component parts above will be inserted.

To insert the to insert the bladder airbag system into the neck gaiter system, at least a portion of the neck and clavicle members of the neck gaiter system, may be removed or separated between the two wicking layers of material where each of the noted component parts above will be inserted.

To insert the digital gyroscope into the neck gaiter system, at least a portion of the neck and clavicle members of the neck gaiter system, may be removed or separated between the two wicking layers of material where each of the noted component parts above will be inserted.

To insert the Bluetooth and sensor arrangement system into the neck gaiter system, at least a portion of the sensor will be next to and touching the wearers skin.

One or more protrusions extending from the cartridge inflator actuation mechanism system, wherein the cartridge contains compressed inert gas, and wherein the cartridge contains a trigger mechanism, and may detachably engage one or more aspects of the entire aspirated inflatable safety protection neck gaiter system and apparatus.

One or more protrusions extending from the impact crash electronic sensor arrangement including a single lead distributed/situated throughout the neck member, and may detachably engage one or more aspects of the entire aspirated inflatable safety protection neck gaiter system and apparatus.

One or more protrusions extending from the digital gyroscope and other sensors arrangement distributed/situated throughout the neck member, and may detachably engage one or more aspects of the entire aspirated inflatable safety protection neck gaiter system and apparatus.

One or more protrusions extending from the bladder airbag system at least a portion of the neck gaiter system, the Bluetooth and sensor system, and may detachably engage one or more aspects of the entire aspirated inflatable safety protection neck gaiter system and apparatus.

The neck gaiter system receiving device when worn independently or with a safety helmet, including the digital gyroscope, electronic sensor system, the single lead or wire, the bladder airbag system, and the compressed gas cartridge actuation mechanism may not present the or a wearer with an abnormal feeling of fit, comfort, or the like. Optimally, the invention will be worn with a safety helmet for sports.

Specific examples of the invention and the structures according to the examples of the invention are described in greater detail below. The reader of the invention should be aware that these specific examples and structures are set forth simply to illustrate the invention, and they should not be construed as limiting the invention.

Some aspects of the present invention relate generally to neck safety protection systems, to protective equipment for safety in sports, and other uses. The bladder airbag system may occupy one or more placements within the neck gaiter portion of the disposable advanced sports safety neck gaiter technology system. The bladder airbag system may occupy one or more placements within the neck gaiter. The inner layer, or closed/sealed tube made of polyurethanes, polyolefins or any polymeric material may occupy one or more placements within the outer breathable and stretchable wicking material layer of the neck gaiter system.

The disposable advanced sports safety neck gaiter technology system may be formed from a variety of comfortable wicking materials already in use in today's market, and may be formed with a variety of characteristics in the prior art.

The inner layer, or closed/sealed tube made of polyurethanes, polyolefins or any polymeric material plastic system may be formed from a variety of materials already in use in today's market, and may be formed with a variety of characteristics.

Example Disposable Advanced Sports Safety Neck Gaiter Technology System. Aspects of the invention relate to safety systems in sports and relates to a disposable advanced sports safety neck gaiter technology system, or a disposable advanced sports safety neck gaiter technology system means any device that a user places on or over some portion of the human body. The advanced sports safety neck gaiter technology system receiving device, (i.e. a disposable advanced sports safety neck gaiter technology system designed to protect the users neck area), which is a disposable advanced sports safety neck gaiter technology system and apparatus including a neck gaiter system with an outer layer and an inner layer, the inner and outer layers including at least a neck gaiter member, at least one member of the neck gaiter member including a digital gyroscope, and at least one member of the neck gaiter member including a sensor arrangement, and at least one member of the neck gaiter member including a lead or wire arrangement, and at least one member of the neck gaiter member including a Bluetooth arrangement and at least one member of the neck gaiter member, and at least one member of the neck gaiter member including a bladder air bag arrangement, and at least one member of the neck gaiter member including a cartridge actuation mechanism and inert gas canister arrangement.

Example Methods of Providing and Using the Disposable Sports Safety Technology Neck Gaiter Device Systems. The neck gaiter system receiving device when worn independently, may not present the user or a wearer with an abnormal feeling of fit, comfort, or the like.

Specific Examples of the Invention. The various figures in the application illustrate examples of a Disposable Advanced Sports Safety Neck Gaiter Technology System apparatus and product. The neck gaiter system in the form of a neck gaiter may be used for warmth and other various uses in today's markets is used as a base shape or design in the examples of the invention.

There may be many modifications to the specifically described structures, systems, and methods of the invention may take place without departing from this invention. As an example, while the invention has been specifically described with respect to specific examples including preferred modes of carrying out the invention, those skilled in the art will appreciate that there may be numerous variations, combinations, and permutations of the above described systems and methods. Furthermore, various specific structural features included in the examples merely represent examples of structural feathers that may be included in some examples of structure according to the invention. Furthermore, with respect to the methods, many variations in the method steps may take place, the steps may be changed in order, various steps or features may be added changes, or omitted, etc., without departing from the invention. Thus, the reader should understand that the spirit and scope of the invention should be construed broadly as set forth in the appended claims.

A disposable aspirated inflatable advanced safety technology neck gaiter protection system and apparatus designed to protect the human neck from injury resulting from sports impacts with a digital gyroscope contained in an interior pocket of the neck gaiter, and also prospectively a crash sensor arrangement in which acceleration, angular, rotational, hyper-extension or hyper-flexion motion resulting from an impact force requires deployment of an apparatus protection device. The method embodies an inherent bladder airbag system that is deployed with resulting impact force and velocity changes on the impact sensor, and changes in pitch, roll, yaw on the gyroscope or the single sensor arrangement using an actuation mechanism and an inflation interconnected bladder system, or tube(s) resulting in release of the inert compressed gas in a cartridge actuation mechanism using a triggering mechanism, which is held in an interior pocket of the neck gaiter resulting in aspiration of the inflatable safety bladder protection neck gaiter.

A disposable advanced sports safety neck gaiter technology system and apparatus that is designed to protect the neck from injury resulting from sports and other impacts. A disposable advanced sports safety neck gaiter in the form of a neck gaiter is a close-fitting garment covering the entire neck, including the front, side(s) and rear of the neck area, typically made of both inner and outer wicking materials, which may contain a digital gyroscope, a single lead crash sensor, a single lead to a sensor, lead(s) or wire(s) for both, a compressed inert gas canister therein for use in sports and daily application for protection.

In accordance with an aspect of the present invention, there is provided a disposable aspirated inflatable safety protection neck gaiter system: an article of a wicking neck gaiter. A neck gaiter is a close-fitting garment covering the whole neck, typically made of wool or other wicking materials for use in sports and daily application for protection from the elements of weather, or for warmth. A disposable aspirated inflatable safety protection neck gaiter system and apparatus including a neck gaiter with an inner layer and an outer layer, the inner and outer layers including at least a neck gaiter member, a neck member, and a clavicle member, at least one member of the neck gaiter member including one or more cavities in the neck portion. A disposable aspirated inflatable safety protection neck gaiter system wherein the one or more apertures in the neck gaiter member, includes one aperture in the top of the neck gaiter for the wearer's head to protrude, and wherein the one or more apertures in the neck gaiter include a aperture in the bottom of the neck gaiter for the wearer's head and shoulders to protrude.

A disposable aspirated inflatable safety protection neck gaiter system wherein the one or more apertures in the neck and clavicle member(s), include one or more apertures include one or more vertical apertures. A disposable aspirated inflatable safety protection neck gaiter system wherein the one or more apertures in the neck and clavicle member(s), include one or more vertical cavities. A disposable aspirated inflatable safety protection neck gaiter system wherein the one or more apertures in the neck and clavicle member(s), include one or more lateral cavities. A disposable aspirated inflatable safety protection neck gaiter system wherein the one or more apertures will not protrude from the neck gaiter system. A disposable aspirated inflatable safety protection neck gaiter system wherein the one or more apertures will not protrude from the neck gaiter system. A disposable aspirated inflatable safety protection neck gaiter system wherein the one or more apertures will not protrude from the neck gaiter system.

A disposable aspirated inflatable safety protection neck gaiter system wherein the one or more apertures in the neck and clavicle member(s) include one or more pockets of the clavicle member of the neck gaiter system, which includes extended side protrusions extending outwardly from the main body of the clavicle member of the neck gaiter system. A disposable aspirated inflatable safety protection neck gaiter system wherein the neck gaiter element includes impact force attenuating elements. A disposable aspirated inflatable safety protection neck gaiter system wherein the neck gaiter element includes impact force sensor and digital gyroscope arrangement. A disposable aspirated inflatable safety protection neck gaiter system wherein the impact force sensor arrangement contains one or more electronic sensor system. A disposable aspirated inflatable safety protection neck gaiter system wherein the electronic sensor system includes Bluetooth and one or more lead(s) or wire(s). A disposable aspirated inflatable safety protection neck gaiter system wherein the neck gaiter element includes a digital gyroscope sensor mechanism.

A disposable aspirated inflatable safety protection neck gaiter system wherein the impact force sensor arrangement contains a sensor system. A disposable aspirated inflatable safety protection neck gaiter system wherein the electronic sensor system includes one or more leads and Bluetooth sensor system. A disposable aspirated inflatable safety protection neck gaiter system wherein the leads include an impact force sensor triggering/actuation mechanism. A disposable aspirated inflatable safety protection neck gaiter system the one or more extended side pocket or protrusions extending outwardly from the main body of the clavicle member of the neck gaiter system includes a cartridge inflator that includes an actuation mechanism. A disposable aspirated inflatable safety protection neck gaiter system the actuation mechanism includes inert compressed gases contained within the cartridge inflator actuation mechanism.

A disposable aspirated inflatable safety protection neck gaiter system the actuation mechanism includes inert compressed gases contained within the cartridge inflator actuation mechanism, and includes an impact force lead or wire attached to the actuation mechanism. A disposable aspirated inflatable safety protection neck gaiter system wherein the leads include a cartridge inflator actuation system. A disposable aspirated inflatable safety protection neck gaiter system wherein the one or more extended side pocket or protrusions extending outwardly from the main body of the clavicle member of the neck gaiter system includes a cartridge inflator that includes an actuation mechanism. A disposable aspirated inflatable safety protection neck gaiter system wherein the actuation mechanism includes a digital gyroscope actuation mechanism. A disposable aspirated inflatable safety protection neck gaiter system wherein the digital gyroscope actuation mechanism includes Bluetooth and/or a lead to an impact or velocity change or angular change sensor mechanism.

A disposable aspirated inflatable safety protection neck gaiter system wherein the impact force attenuation neck gaiter and clavicle system(s) include a bladder airbag system in the neck and clavicle members of the neck gaiter system. A disposable aspirated inflatable safety protection neck gaiter system wherein the digital gyroscope sensor system determines there is a sufficient pre-determined change in the wearer's positioning relative to the ground due to acceleration, angular, rotational, extension or flexion motion resulting from over rotation or unintended falling off of equipment or animals requires deployment of an apparatus protection device of sufficient magnitude, it sends a signal to the cartridge inflator actuation system. The inflator sets off a chemical charge, producing an explosion of compressed inert gas, filling up the bladder airbag system.

A disposable aspirated inflatable safety protection neck gaiter system the digital gyroscope sensor system is activated by the sensor system. A disposable aspirated inflatable safety protection neck gaiter system the impact sensor system crash electronic sensor(s) determines there is an impact, velocity or angular change of sufficient magnitude, it sends a signal to the cartridge inflator actuation system. The inflator sets off a chemical charge, producing an explosion of compressed inert gas, filling up the bladder airbag system.

A disposable aspirated inflatable safety protection neck gaiter system, comprising: an article of neck gaiter including one continuous member comprised of a neck gaiter member, a neck member and a clavicle member; the neck and clavicle member(s) include a defining cavity, or pocket, wherein the defining cavity includes a cartridge inflator actuation system, wherein the cartridge contains compressed inert gas, and wherein the cartridge contains a trigger mechanism; the neck gaiter member including one or more apertures to fit over the wearer's head to be seated comfortably over and around the wearer's neck area; the neck gaiter member includes an impact crash electronic sensor arrangement including either a Bluetooth or a single lead(s) situated at the base or clavicle of the neck gaiter member; the neck member including a digital gyroscope sensor, wherein the neck member includes one or more lead(s) in the neck member, and wherein the neck member includes one or more mechanisms in the neck member, wherein the neck member includes a bladder airbag system, and wherein the neck gaiter includes one or more pockets in the inner wicking layer to contain the inert gas canister and one or more pockets to contain the digital gyroscope sensor mechanism.

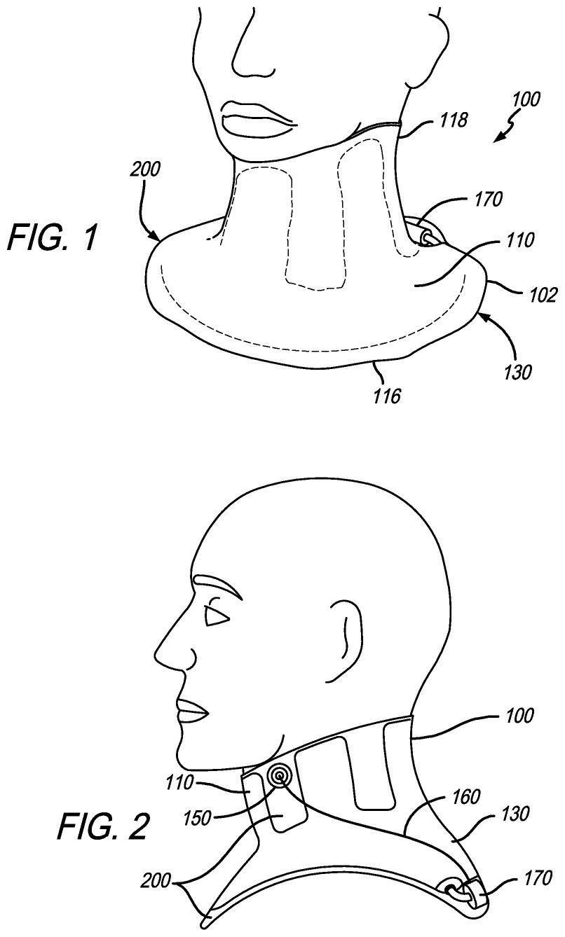

Referring now to the drawings, wherein the showings are for purposes of illustrating the present invention and not for purposes of limiting the same, FIGS. 1-5 show a neck gaiter system 100 for protecting a wearer's neck during dangerous activities. For example, the present invention can be used to protect a user while horseback riding, or riding an ATV or motorcycle.

In a preferred embodiment, the present invention includes a gyro sensor, gyroscope and/or other sensors that can sense rotation, velocity changes, impact force, pitch, roll, yaw changes. In horseback, ATV, or motorcycle riding as examples, if a person is thrown from their mount there is a possibility of landing on their head and injuring their neck. The present invention senses the sudden rotation or change in angle of the rider as they go from generally vertical (i.e., perpendicular to the ground), and past horizontal (i.e., parallel to the ground) as they fall to the ground. In a preferred embodiment, once the gyro sensor senses a predetermined angle change (i.e., past 45°), the air bag is inflated to protect the user's neck. As used herein, the term gyro sensor or digital gyroscope system is used to represent any type of sensor or gyroscope that can sense the rotational or angular change described above.

FIG. 1 illustrates the neck gaiter system 100, which includes an inner layer 130 and an outer layer 200 and shows placement on the user or wearer's neck. FIG. 1 also illustrates the bladder air bag system 110, the inert gas canister 170, and the double wicking material for both the inner 130 and outer layers 200. It will be appreciated that the inner and outer layers can be made of non-wicking materials.

FIG. 2 shows the interior wicking layers 130 (with the outer layer removed) with a bladder air bag system 110, an inert gas canister 170 with a single lead 160 from an impact and/or velocity crash sensor 150 that may also include a Bluetooth sensor system, and may also include the gyro system. The sensor 150 may either be in between the wicking layers, or located next to and touching the wearers neck/skin.

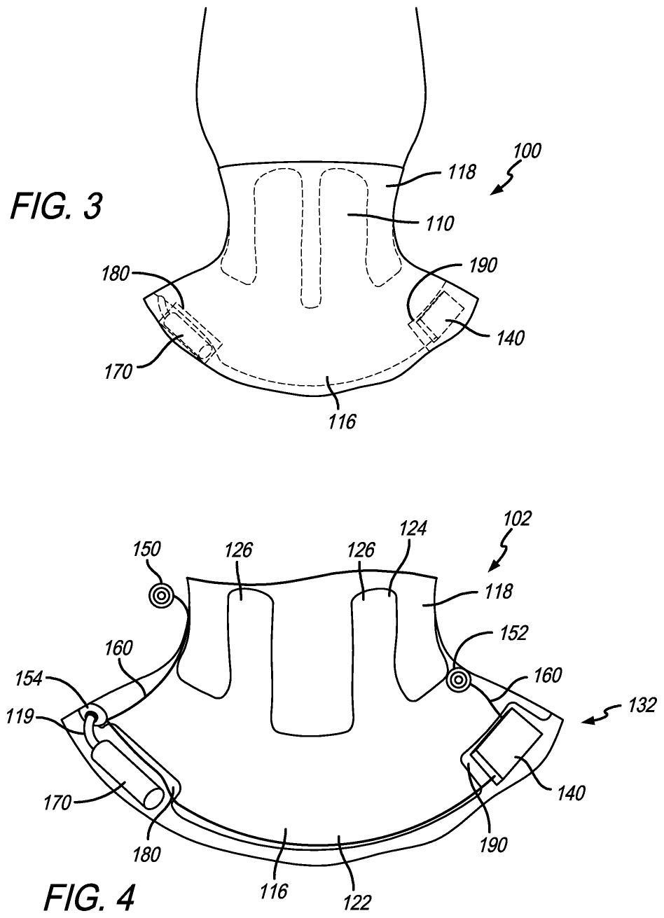

FIG. 3 shows a rear view of the neck gaiter system, including the bladder air bag system 110, the inert gas canister 170 contained within a pocket or chamber 180 in the double wicking material, and a digital gyroscope or gyro sensor 140 contained within a pocket or chamber 190. The pockets can be created in one of the inner or outer layers or can be formed between the two layers via stitching or the like.

In FIG. 3, the inert gas cartridge or canister 170 is shown using dotted lines, as it appears in the example of a neck gaiter system 100 including a cavity or pocket for the insertion of the cartridge actuation system 170 connected to the trigger mechanism 154 (see FIG. 4), which is held in an interior pocket 180 of the gaiter member 102 resulting in aspiration of the inflatable safety bladder 110 protection neck gaiter in accordance with the invention because the actuation system 170 is in fluid communication (via a conduit 119) with the bladder 110. Furthermore, as shown in FIG. 4, the bladder 110 includes a clavicle portion 122, a neck portion 124 and a plurality of chambers 126 extending upwardly from the clavicle portion 116 of the gaiter member 102 to the neck portion 118 of the gaiter member 102. The clavicle portion 122 of the bladder 110 is ring shaped. The neck portion of 122 may also be ring shaped.

FIG. 4 shows a rear view of the gaiter member with the outer layer removed, and includes the bladder air bag system 110, inert gas canister 170 contained within the pocket 180 in the double wicking material, a single lead 160, and the digital gyroscope 140 contained within pocket 190 in the double wicking material, with a single lead 160 from a sensor 152, and an impact and/or velocity crash sensor 150 that may also include a Bluetooth sensor system, and may also include the gyro system. The sensor 150 may either be in between the wicking layers, or located next to and touching the wearers neck/skin.

In a preferred embodiment, the neck gaiter system 100 includes the gaiter member 102 that includes a neck portion 118 and a clavicle portion 116, a sensor system 132 that includes at least one gyro sensor 140 positioned on the gaiter member 102, an actuation system 170 in electrical communication with the sensor system 132, and a bladder 110 positioned on at least the neck portion 118 of the gaiter member 102. The bladder 110 is in fluid communication with the actuation system 170. The gaiter member 102 is configured to be received on the neck of a wearer. In use, when the sensor system 132 senses a predetermined rotational change, the actuation system 170 inflates the bladder. The rotational change can be measured from an orientation perpendicular to the ground (where a person is standing or seated upright) through 180° where the person is upside down head first toward the ground. The rotational change (the angle at which the actuation system is triggered) can be any angular change or angle between 1° and 180°. Other ranges can be any set of angles between 45° and 180° or between 90° and 180° (i.e., the bladder is inflated after a 45° or 90° angle is detected or sensed). This angle can be in any direction. In other words, the angle can be based on a twisting or spinning rotation. The sensing system can also be configured to sense other movements or events, such as linear acceleration, angular acceleration, rotational acceleration, extension or flexion, that trigger the inflation of the bladder. Sensor 150 can be configured to sense any of these movements. The sensing system can also be configured such that the airbag is inflated after two factors are sensed. For example, if the rotational change angle is set at 45°, a user will not want the bladder inflating if they lean over on the horse past a 45° angle to speak with someone. Accordingly, the sensor system can be configured to inflate the bladder if both the gyro sensor senses a rotational change past the predetermined angle and the sensor system senses a predetermined velocity, acceleration or other factor (e.g., when the horse bucks the rider).

In a preferred embodiment, the sensor system includes a body sensor 152 in communication with the gyro sensor 140. The body sensor 152 can be any sensor that senses that a person is wearing the neck gaiter system. The body sensor 152 can sense temperature, skin contact or other characteristic indicating that the system is being worn by a user. For example, when the body sensor 132 senses a predetermined temperature or it senses skin contact, the gyro sensor 140 switched from a deactivated mode to an activated mode, such that it can be activated if the predetermined requirements or factors discussed herein are met. The body sensor can be located anywhere within the gaiter member 102. In a preferred embodiment, the body sensor 132 is located on the inner layer 130 such that it can make contact with the user's skin.

In a preferred embodiment, the sensor system can also include biometric, impact, velocity and gyro sensors if required. These sensors can be any sensor that senses any change of velocity, impact force, pitch, roll, yaw to a person who is wearing the neck gaiter system. The biometric, impact, velocity and gyro sensor(s) 150 can sense skin contact or other characteristic indicating that the system is being worn by a user. For example, when any one or more of the sensor(s) senses an unwelcome or predetermine change in impact force, velocity, or pitch, roll, yaw, the sensor system triggers the bladder to inflate.

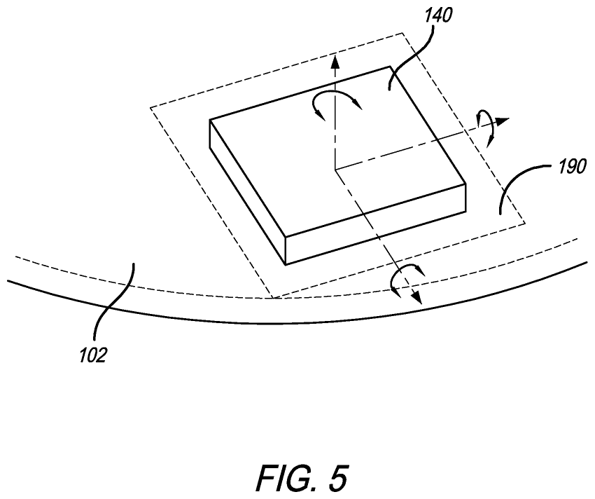

FIG. 5 shows a close up of the gyro sensor 140 and includes indicating that it, for example, can sense roll, pitch and yaw rotational motion.

An overall description, as described above, generally illustrates a front exterior view of the example of the invention in which this invention relates generally to a disposable advanced sports safety neck gaiter technology system 100 designed to protect the human neck from injury resulting from sports impacts with a bladder air bag arrangement 110 in which velocity of angular, rotational, and linear acceleration resulting from an impact force requires dissipation and energy attenuation. The method embodies an inherent inner layer system 130 that is enclosed within the outer layer 200, and that the inner layer 130 contains the bladder air bag system 110, along with the digital gyroscope mechanism 140, with the sensor 150, and the sensor lead and wire 160, and the inert canister 170 that is part of the actuation mechanism sealed within the durable inner closed/sealed tube. The method also embodies a pocket to hold the inert gas canister 170, and a pocket to hold the digital gyroscope 140.

The invention also includes in the neck gaiter system 100 comprised of breathable double fabric knit worn around/over the neck, a bladder air bag system 110 arranged with specific characteristics around the entirety of the neck to support when there is an impact to the head, a digital gyroscope 140, and an inner layer system 130, outer layer system 200, and an inner layer comprised of wicking material, an outer layer comprised of wicking material, which also may contain a body sensor 152, and may also contain a sensor lead and wire 160, and the inert canister 170 that is linked to the actuation mechanism.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

The teachings of the disclosure provided herein can be applied to other systems, not necessarily the system described above. The elements and acts of the various embodiments described above can be combined to provide further embodiments. Any measurements described or used herein are merely exemplary and not a limitation on the present invention. Other measurements can be used. Further, any specific materials noted herein are only examples: alternative implementations may employ differing materials.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A neck gaiter system comprising:
a gaiter member that includes a neck portion and a clavicle portion, wherein the gaiter member is configured to be received on the neck of a wearer when worn,
a sensor system that includes at least one gyro sensor positioned on the gaiter member,
an actuation system in electrical communication with the sensor system, and
a bladder positioned on at least the neck portion of the gaiter member, wherein the bladder is in fluid communication with the actuation system,
wherein when the sensor system senses a predetermined rotational change, the actuation system inflates the bladder.

2. The neck gaiter system of claim 1 wherein the sensor system includes an accelerometer.

3. The neck gaiter system of claim 1 wherein the sensor system includes a biometric sensor.

4. The neck gaiter system of claim 3 wherein the biometric sensor is positioned to contact the wearer's skin.

5. The neck gaiter system of claim 1 wherein the gaiter member includes an inner layer and an outer layer, and wherein the gyro sensor is positioned between the inner and outer layers.

6. The neck gaiter system of claim 5 wherein the bladder is positioned between the inner and outer layers.

7. The neck gaiter system of claim 1 wherein the actuation system is positioned on the clavicle portion of the gaiter member.

8. The neck gaiter system of claim 1 wherein the bladder includes a clavicle portion and a neck portion, wherein the bladder includes a plurality of chambers extending upwardly from the clavicle portion to the neck portion.

9. The neck gaiter system of claim 8 wherein the clavicle portion of the bladder is ring shaped.

10. The neck gaiter system of claim 1 wherein the actuation system is in electrical communication with the sensor system via a plurality of leads, and wherein the leads are positioned between the first and second layers.

11. The neck gaiter system of claim 2 wherein when the sensor system senses the predetermined rotational change and a predetermined velocity change, the actuation system inflates the bladder.

12. The neck gaiter system of claim 1 wherein the sensor system includes a Bluetooth sensor.

13. The neck gaiter system of claim 1 wherein the sensor system includes a GPS sensor.

14. The neck gaiter system of claim 8 wherein the neck portion of the bladder is ring shaped.
